(19) United States
(12) Patent Application Publication
(10) Pub. No.: US 2026/0250698 A1
(43) Pub. Date: Aug. 27, 2026

(54) PHYLLOSTACHYS EDULIS-DERIVED PheXTH2 PROTEIN FOR REGULATING CULM CELL WALL THICKNESS OF PLANT, AND ENCODING GENE AND USE THEREOF

(71) Applicant: International Centre for Bamboo and Rattan, Beijing (CN)

(72) Inventors: Jian GAO, Beijing (CN); Yali XIE, Beijing (CN)

(73) Assignee: International Centre for Bamboo and Rattan, Beijing (CN)

(21) Appl. No.: 19/270,528

(22) Filed: Jul. 16, 2025

(30) Foreign Application Priority Data
Feb. 26, 2025 (CN) 202510216662.0

(51) Int. Cl. C12N 15/82; C07K 14/415

(52) U.S. Cl. CPC C12N 15/8242; C07K 14/415

(57) ABSTRACT

A Phyllostachys edulis-derived PheXTH2 protein for regulating a culm cell wall thickness of a plant, and an encoding gene and use thereof are provided. Through Agrobacterium-mediated transformation of wild-type Arabidopsis thaliana with a gene expression vector, overexpression of a PheXTH2 gene is found to increase cell wall thickness in interfascicular fibers. This result demonstrates that the gene exhibits a regulatory function in the culm cell wall thickness of a plant. Therefore, PheXTH2 serves as a potential molecular breeding tool for improving bamboo culm wood properties to enhance lumber yield and ecological benefits, indicating broad application prospects.

### CROSS-REFERENCE TO RELATED APPLICATION

This application is based upon and claims priority to Chinese Patent Application No. 202510216662.0, filed on Feb. 26, 2025, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named WGJB0274_Sequence_Listing.xml, created on Jul. 3, 2025, and is 7,292 bytes in size.

### TECHNICAL FIELD

A Phyllostachys edulis-derived PheXTH2 protein for regulating a culm cell wall thickness of a plant, and an encoding gene and use thereof are provided, relating to the technical field of plant molecular biology.

### BACKGROUND

Bamboo is a natural composite material renowned for its rapid growth and exceptional mechanical properties. Phyllostachys edulis, though taxonomically a grass, develops highly lignified stems. The wood properties of plants are determined by cell wall thickness and composition, while understanding of the molecular regulatory mechanisms governing culm cell wall development remains limited.

Xyloglucan endotransglycosylase/hydrolases (XTH) enzymes mediate cell wall loosening by catalyzing the hydrolysis and re-ligation of xyloglucan molecules between cellulose microfibrils (a process termed “molecular grafting”). Specific XTH genes play defined roles in plant development: AtXTH4 and AtXTH9 facilitate wood cell expansion and secondary wall formation in Arabidopsis, while SbHY5 regulates cell wall development in sweet sorghum (Sorghum bicolor) by modulating hemicellulose content through SbXTH7. These studies collectively demonstrate XTH showing the capacity to orchestrate cell wall biosynthesis.

Consequently, isolating and characterizing XTH genes associated with culm cell wall development in Phyllostachys edulis is critical for comprehensively elucidating the molecular mechanisms underlying cell wall formation of Phyllostachys edulis. This represents an urgent technical challenge requiring resolution in the field.

### SUMMARY

In order to solve the above technical problems, the present disclosure provides a Phyllostachys edulis-derived PheXTH2 protein having the amino acid sequence set forth in SEQ ID NO: 1.

The present disclosure further provides a nucleic acid encoding the Phyllostachys edulis-derived PheXTH2 protein.

Preferably, the nucleic acid has the nucleotide sequence set forth in SEQ ID NO: 2.

The present disclosure further provides a biological material, including the Phyllostachys edulis-derived PheXTH2 protein or the nucleic acid; where the biological material is selected from the group consisting of a recombinant DNA, an expression cassette, a transposon, a plasmid vector, a viral vector, an engineered bacterium, and a non-regenerable plant cell or tissue.

The present disclosure further provides the use of the Phyllostachys edulis-derived PheXTH2 protein, the nucleic acid, or the biological material in regulating a culm cell wall thickness of a plant.

Preferably, the culm cell wall of the plant is an interfascicular fiber cell wall.

In specific embodiments, the expression level or activity of the Phyllostachys edulis-derived PheXTH2 protein is enhanced to increase culm cell wall thickness of the plant.

Preferably, a Phyllostachys edulis-derived PheXTH2 gene is overexpressed to increase culm cell wall thickness of the plant.

In some embodiments, the overexpression is achieved through one or a combination of more approaches selected from (1) to (5):
(1) introducing a plasmid containing the gene;
(2) increasing a copy number of the gene on a plant chromosome;
(3) modifying a promoter sequence of the gene on the plant chromosome;
(4) operably linking a strong promoter to the gene; and
(5) introducing an enhancer.

In specific embodiments, the expression level or activity of the Phyllostachys edulis-derived PheXTH2 protein is reduced or eliminated to decrease culm cell wall thickness of the plant.

Preferably, the Phyllostachys edulis-derived PheXTH2 gene is knocked out to decrease culm cell wall thickness of the plant.

In some embodiments, the knockout is conducted using any conventional gene knockout technique.

In some embodiments, the knockout is conducted via homologous recombination.

In some embodiments, the knockout is conducted using CRISPR-Cas system editing.

The present disclosure further provides the use of the Phyllostachys edulis-derived PheXTH2 protein, the nucleic acid, or the biological material in the preparation of a transgenic plant.

In some embodiments, the use includes the following steps:

Cloning primers are designed based on the CDS sequence of the PheXTH2 gene. PCR amplification is conducted using cDNA obtained by reverse transcription of total RNA from Phyllostachys edulis stems as a template. The amplification product is ligated to a pMD19-T cloning vector, followed by transformation into Escherichia coli (DH5α) for sequencing. An overexpression vector pCAMBIA-2300-eGFP is constructed with the sequence-confirmed insert. Arabidopsis thaliana is transformed via Agrobacterium-mediated transformation to obtain the transgenic plant.

The present disclosure further provides the use of the Phyllostachys edulis-derived PheXTH2 protein, the nucleic acid, or the biological material in plant breeding.

Breeding methods include, but are not limited to: genetic transformation, hybridization, backcrossing, selfing, or vegetative propagation.

The present disclosure further provides a method for regulating a culm cell wall thickness of a plant, including: enhancing an expression level or an activity of the Phyllostachys edulis-derived PheXTH2 protein or an encoding gene thereof in the plant by genetic engineering to increase the culm cell wall thickness of the plant; alternatively, reducing or eliminating the expression level or the activity of the Phyllostachys edulis-derived PheXTH2 protein or the encoding gene thereof in the plant by the genetic engineering to decrease the culm cell wall thickness of the plant.

Preferably, the genetic engineering includes: introducing an expression vector carrying the target gene into plant cells using Ti plasmid, plant viral vector, direct DNA transformation, microinjection, or electroporation.

Preferably, the method further includes: identifying positive transgenic plants through kanamycin (Kan) resistance screening; observing and detecting phenotypes of positive transgenic plants; analyzing target gene expression in transgenic and wild-type plants via semi-quantitative PCR; preparing 8-μm sections of stems using paraffin sectioning; staining the sections with toluidine blue; observing cell walls under an optical microscope; and quantifying the cell wall thickness.

Preferably, the plant is selected from the group consisting of Arabidopsis thaliana and

Phyllostachys edulis.

Compared with the prior art, the present disclosure has the following beneficial effects: The present disclosure provides for the first time a Phyllostachys edulis-derived PheXTH2 protein associated with cell wall development, and an encoding gene and use thereof. Through Agrobacterium-mediated transformation of wild-type Arabidopsis thaliana with a gene expression vector, overexpression of a PheXTH2 gene is found to increase cell wall thickness in interfascicular fibers. This result demonstrates that the gene exhibits a regulatory function in the cell wall thickness. Consequently, PheXTH2 serves as a potential molecular breeding tool for improving the wood properties of the bamboo culm to enhance lumber yield and ecological benefits, indicating broad application prospects.

### BRIEF DESCRIPTION OF THE DRAWINGS

FIG. 1 shows detection results of positive transgenic Arabidopsis thaliana plants overexpressing the PheXTH2 gene; where symbols are M: DNA Marker; WT: Wild-type Arabidopsis thaliana (Col-0), negative control; P: pCAMBIA-2300-eGFP plasmid, negative control; and 2 #: PheXTH2; H₂O: Negative control;

FIG. 2 shows culm cell wall thickness measurements in Arabidopsis thaliana after PheXTH2 overexpression; where different lowercase letters indicate statistically significant differences; and

FIG. 3 shows the semi-quantitative analysis of PheXTH2 gene overexpression in Arabidopsis thaliana; where symbols are M: DNA Marker; WT: Wild-type Arabidopsis thaliana (Col-0), negative control; Actin: Reference gene; and XTH2: PheXTH2 gene.

### DETAILED DESCRIPTION OF THE EMBODIMENTS

In order to make the objectives, technical solutions, and advantages of the examples of the present disclosure clearer, the technical solutions in the present disclosure are described clearly and completely below. Apparently, the described examples are some rather than all of the examples of the present disclosure. All other examples obtained by those of ordinary skill in the art based on the examples of the present disclosure without creative efforts shall fall within the protection scope of the present disclosure. In the examples provided in this specification, where specific techniques or conditions are not specified, the procedures follow techniques or conditions described in literature within the field, such as Molecular Cloning: A Laboratory Manual (Sambrook J & Russell D. W., 2001) or manufacturer's instructions. Reagents or instruments not specified by manufacturers are all conventional products that can be purchased through formal channels.

### EXAMPLE

### I. Cloning of the Phyllostachys edulis-Derived PheXTH2 Gene and Vector Construction

1. Phyllostachys edulis Culm Materials were Collected from Guangde City, Anhui Province (N30° 52′33″8, E119°25′22″4). The Samples were Transported to the Laboratory on Dry Ice and Stored at −80° C. For Subsequent Experiments.

### 2. RNA Extraction

The procedure was conducted as follows:
(1) Approximately 2-3 g of Phyllostachys edulis culm tissue was placed in a mortar pre-chilled with liquid nitrogen, ground to powder in liquid nitrogen, and transferred into a 2 mL RNase-free centrifuge tube pre-cooled in liquid nitrogen.
(2) 1 mL Trizol was added to each tube, followed by immediate inversion mixing.
(3) 200 μL chloroform was added to each tube, vortexed for 1 min, and incubated on ice for 15 min.
(4) Centrifugation was conducted at 12,000 rpm for 10 min at 4° C.
(5) 600 μL of supernatant was transferred to a 1.5 mL RNase-free tube. An equal volume of isopropanol was added, and the mixture was incubated on ice for 15 min.
(6) Centrifugation was conducted at 12,000 rpm for 10 min. The supernatant was discarded, and the pellet was washed 2 times with 70% ethanol (prepared with nuclease-free water).
(7) After air-drying at room temperature, the pellet was dissolved in 50-100 μL nuclease-free water.
   3. Conversion of Total RNA to cDNA Via Reverse Transcription
(1) The first-strand cDNA synthesis was conducted using the PrimeScript™ RT Reagent Kit with gDNA Eraser (TaKaRa) according to the manufacturer's protocol:
   (a) Reaction for gDNA Removal

The reaction mixtures were prepared on ice with components listed in Table 1. PCR protocol was: 42° C. for 2 min.

TABLE 1
Reaction system for gDNA removal

| Reagent | Volume | |
|---|---|---|
| 5 × gDNA Eraser Buffer | 2.0 | μL |
| gDNA Eraser | 1.0 | μL |
| Total RNA | 3.0 | μL |

| | |
|---|---|
| RNase-free water dH₂O | making up to 10 μL |

### (b) Reverse Transcription Reaction

The reverse transcription PCR protocol was: 37° C. for 15 min; 85° C. for 5 s; and storage at 4° C. The reaction system was prepared as shown in Table 2.

TABLE 2
Reverse transcription reaction system

| Reagent | Volume | |
|---|---|---|
| Reaction solution in step (a) | 10.0 | μL |
| PrimeScript Enzyme Mix I | 1.0 | μL |
| RT primer Mix | 1.0 | μL |
| 5 × PrimeScript Buffer | 4.0 | μL |
| RNase-free water | 4.0 | μL |
| Total | 20 | μL |

### 4. Gene Cloning

(1) The nucleotide sequence of the PheXTH2 gene was set forth in SEQ ID NO: 2, and the amino acid sequence of its encoded protein was set forth in SEQ ID NO: 1.

Primer sequences were as follows:
PheXTH2-F: ATGAGGACGATTGCGGTTGG (SEQ ID NO: 3)
PheXTH2-R: TCAACGCAGCTTGCACTCG (SEQ ID NO: 4)
(2) PCR Amplification

Using cDNA from Phyllostachys edulis as the template, PCR amplification was conducted with the corresponding primers. The reaction program was: 94° C. for 1 min; 30 cycles of 95° C. for 10 s, 60° C. for 30 s, and 72° C. for 1 min; followed by 72° C. for 10 min. A 20 μL amplification system was prepared on ice as shown in Table 3.

TABLE 3
PCR reaction system

| Reagent | Volume | |
|---|---|---|
| 2 × Tap PCR mix | 10.0 | μL |
| Upstream primer | 1.0 | μL |
| Downstream primer | 1.0 | μL |
| Template | 2 | μL |
| ddH₂O | 6 | μL |
| Total | 20.0 | μL |

(3) 1% agarose gel electrophoresis was conducted to detect whether the band size matched the expected size. Bands of the expected size were then subjected to gel extraction and purification for the next step.
(4) Gel extraction and purification were conducted according to the instructions of the M5 Gel Extraction Kit (Mei5Bio, Beijing).

### 5. Ligation of Cloning Products to Vector

(1) In a microcentrifuge tube, 100 ng of PCR product, 1 μL Pmd19-T Vector (TaKaRa), 5 μL 2× Ligation Buffer (TaKaRa), and sufficient ddH₂O were added to reach a total volume of 10 μL. The mixture was gently mixed and incubated at 16° C. for 30 min.
(2) The ligation product was transformed into competent E. coli cells (DH5α). After 30-min incubation on ice, heat shock was applied at 42° C. for 30-60 s, followed immediately by 3-min incubation on ice. 600 μL LB liquid medium was added, and cells were recovered at 37° C. for 30 min. The mixture was spread-plated onto agar plates containing the appropriate antibiotic.
(3) Following overnight incubation, well-grown colonies were selected for PCR verification of target gene insertion. Sequence-confirmed positive clones were submitted to Azenta (Suzhou) for sequencing.

### 6. Construction of Expression Vector

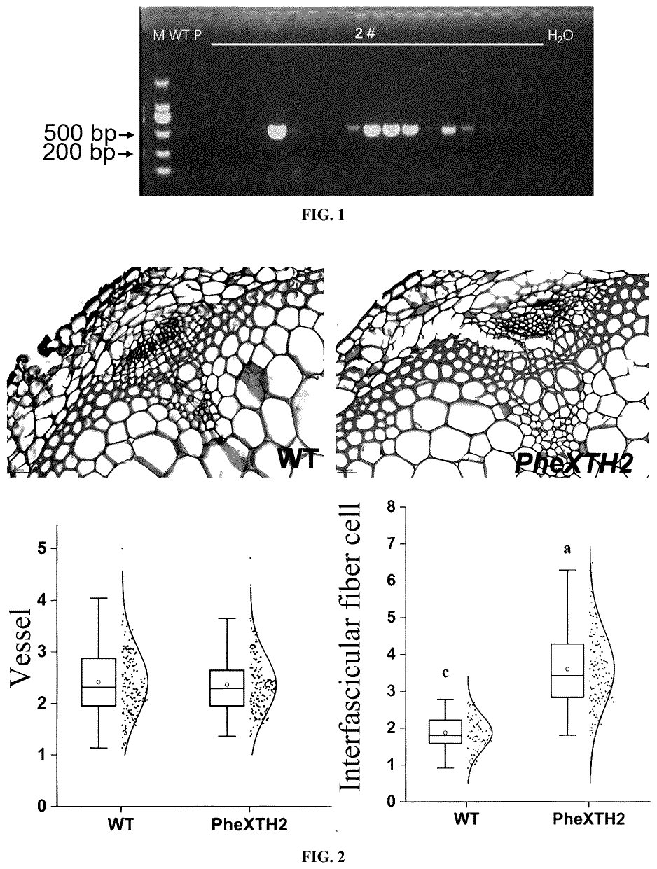

(1) Homologous arms of the pCAMBIA-2300-eGFP vector were added to the sequence-verified PheXTH2 plasmid via PCR amplification. The vector and target gene were ligated following the protocol of the M5™ SuperFast Seamless Cloning Kit (Mei5Bio).
(2) The ligation product was transformed into E. coli competent cells (DH5α) using the heat shock method. Transformed bacteria were spread-plated onto LB agar plates containing appropriate selective antibiotics.
(3) Bacteria were cultured until visible colonies formed. Single colonies were selected for colony PCR, and positive clones were sequenced. Successful construction of the pCAMBIA-2300-PheXTH2-eGFP overexpression vector was confirmed by sequence verification.
   7. Plant transformation
(1) Robust Arabidopsis thaliana plants were grown under long-day conditions (14-h light/10-h dark) for approximately 5 weeks.
(2) The primary inflorescence (1-5 cm) was excised to promote lateral inflorescence development. Floral dip transformation was conducted within 3-5 d post-trimming. Plants were thoroughly watered 1 d prior to transformation to ensure stomatal opening.
(3) The pCAMBIA-2300-PheXTH2-eGFP vector was transformed into Agrobacterium tumefaciens strain GV3101. Positive monoclonal colonies were identified by PCR, inoculated into 1 mL LB medium with antibiotics, and cultured at 28° C. for 16 h. Then, 150 μL culture was transferred to 150 mL fresh LB medium and shaken at 28° C. until OD₆₀₀>0.8.
(4) Bacterial cells were pelleted by centrifugation at 4,000×g for 20 min and resuspended in 120 mL infiltration medium (10% sucrose+400 μL/L Silwet-77, mixed thoroughly pre-dipping, OD₆₀₀=0.8-1.0). Concurrently, siliques and fully opened flowers were removed from plants.
(5) Inflorescences were immersed in bacterial suspension for 45 s with gentle agitation.
(6) Infected plants were covered for dark incubation (48-72 h), and then transferred to normal light conditions with periodic reinfection.
(7) Plants were cultivated to maturity. Seeds were harvested, and dried for about 1 week, and transformants were screened.
(8) Arabidopsis thaliana genomic DNA was extracted using the CTAB method. PCR screening was performed to identify positive transformants (FIG. 1).

### 8. Observation and Measurement of Culm Cell Wall Thickness

Positive transgenic Arabidopsis thaliana plants confirmed by genetic screening were selected as an experimental group, with untransformed wild-type Arabidopsis thaliana plants (Col-0) serving as a control. All plants were cultivated under identical conditions to ensure consistency. When inflorescence stems reached not less than 20 cm in height, 2-cm basal segments of Arabidopsis thaliana were excised and fixed in 70% FAA at 4° C. for 48 h. Samples then underwent: gradient ethanol dehydration, xylene clearing, and paraffin embedding. Embedded samples were sectioned at 8 μm thickness and stained with toluidine blue. After being mounted. Cell walls were examined under an Olympus optical microscope. Micrographs were analyzed to quantify cell wall thickness in vessels and interfascicular fibers, followed by statistical analysis and graphical representation.

FIG. 2 shows differential cell wall phenotypes between PheXTH2-overexpressing and wild-type (WT) Arabidopsis thaliana. While vessel cell wall thickness remained unchanged, interfascicular fibers exhibited significantly increased thickness in PheXTH2-overexpressing plants, indicating that PheXTH2 promotes thickening of interfascicular fiber cell walls.

### 9. Semi-Quantitative Gene Expression Analysis

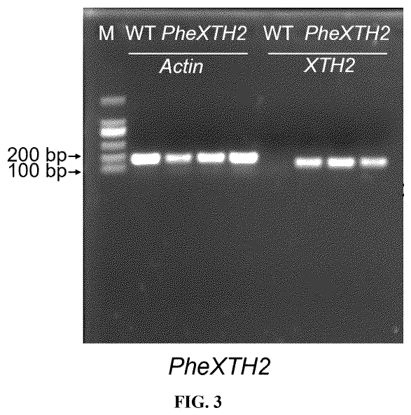

Semi-quantitative analysis of gene expression was conducted using Arabidopsis thaliana Actin as the reference gene (control group). The PheXTH2 gene was amplified by PCR and subsequently analyzed via agarose gel electrophoresis. Results demonstrated that compared with controls, PheXTH2 expression was significantly elevated following overexpression (FIG. 3).

Finally, it should be noted that the foregoing embodiments are only used to illustrate the technical solutions of the present disclosure, and are not intended to limit the present disclosure. Although the present disclosure is described in detail with reference to the foregoing embodiments, those of ordinary skill in the art should understand that they can still modify the technical solutions described in the foregoing embodiments, or make equivalent substitutions to some technical features therein. These modifications or substitutions do not make the essence of the corresponding technical solutions depart from the spirit and scope of the technical solutions in the embodiments of the present disclosure.

What is claimed is:

1. A method for generating a transgenic plant, comprising upregulating or downregulating a gene encoding a Phyllostachys edulis-derived PheXTH2 protein in a plant;
wherein the Phyllostachys edulis-derived PheXTH2 protein has the amino acid sequence set forth in SEQ ID NO: 1, and the gene has the nucleotide sequence set forth in SEQ ID NO: 2.

2. The method according to claim 1, wherein the plant is selected from the group consisting of Arabidopsis thaliana and Phyllostachys edulis.

3. A method for breeding a plant, comprising upregulating or downregulating a gene encoding a Phyllostachys edulis-derived PheXTH2 protein;
wherein the Phyllostachys edulis-derived PheXTH2 protein has the amino acid sequence set forth in SEQ ID NO: 1, and the gene has the nucleotide sequence set forth in SEQ ID NO: 2.

4. The method according to claim 3, wherein the plant is selected from the group consisting of Arabidopsis thaliana and Phyllostachys edulis.

5. A method for regulating a culm cell wall thickness of a plant, comprising: regulating an expression level or an activity of a Phyllostachys edulis-derived PheXTH2 protein or an encoding gene thereof in the plant by genetic engineering.

6. The method according to claim 5, wherein the regulating the expression level or the activity of the Phyllostachys edulis-derived PheXTH2 protein or the encoding gene thereof in the plant by genetic engineering comprises enhancing the expression level or the activity of the Phyllostachys edulis-derived PheXTH2 protein or the encoding gene thereof in the plant by genetic engineering to increase the culm cell wall thickness of the plant.

7. The method according to claim 5, wherein the regulating the expression level or the activity of the Phyllostachys edulis-derived PheXTH2 protein or the encoding gene thereof in the plant by genetic engineering comprises reducing or eliminating the expression level or the activity of the Phyllostachys edulis-derived PheXTH2 protein or the encoding gene thereof in the plant by the genetic engineering to decrease the culm cell wall thickness of the plant.

8. The method according to claim 5, wherein the Phyllostachys edulis-derived PheXTH2 protein has the amino acid sequence set forth in SEQ ID NO: 1.

9. The method according to claim 5, wherein the encoding gene has the nucleotide sequence set forth in SEQ ID NO: 2.

10. The method according to claim 5, wherein the plant is selected from the group consisting of Arabidopsis thaliana and Phyllostachys edulis.


SEQUENCE LISTING (4 sequences)

SEQ ID NO: 1 - protein, 290 aa, Phyllostachys edulis
  REGION 1..290: note = amino acid sequence of Phyllostachys edulis-derived PheXTH2 protein
MRTIAVGIVAMACLVAISQGGNFYQDAEMSWGQGRGKIVDGGRGLDLTLDRSSGSGFQSKNEYLFGKIDMQIKLVPGNSAGTVTTFYLSSQGSTHDEIDFEFLGNVTGEPYTLHTNVFTQGQGQREQQFRLWFDPTNAFHTYSIIWNPQHVIFAVDGTPIRDFKNLEARGVAFPKSQPMRLYASLWNADDWATQGGRVKADWSRAPFVASFRNFNADACVWSAGAQRCPVGTMEAAAAAPGVSRSSWWNQQLSDMSYRRMRWVQRKFMIYNYCTDRKRFPQGVPAECKLR

SEQ ID NO: 2 - DNA, 873 bp, synthetic construct
  misc_feature 1..873: note = nucleotide sequence encoding the Phyllostachys edulis-derived PheXTH2 protein
  source 1..873: mol_type = other DNA
atgaggacgattgcggttggcattgtggcgatggcctgcttagtggccatctcgcagggtggcaacttctaccaggacgccgagatgtcgtgggggcagggccgcggcaagatcgtggacggcggccgcgggctcgacctcaccctggacaggagctccgggtcagggttccagtccaagaacgagtacctcttcggcaagatcgacatgcagattaagctcgtcccgggcaactccgccggcactgtcaccaccttctatttgtcgtcgcaggggtcgacgcacgacgagatcgacttcgagttcctgggcaacgtgaccggcgagccgtacacgctgcacaccaacgtgttcacgcaggggcagggacagcgggagcagcagttccggctctggttcgaccccaccaatgccttccacacctactccatcatctggaacccgcagcatgtcatatttgccgtggacggcaccccgatcagggatttcaagaacctcgaggcgcgcggcgtggcgttccccaagagccagccgatgcggctgtacgcgagcctgtggaacgcggacgactgggccacgcagggcgggcgggtgaaggcggactggagccgggcgccgttcgtcgcctcgttccgcaacttcaacgccgacgcgtgcgtgtggtcggccggcgcgcagcggtgccccgtcggcacgatggaggcggccgcggcggcgcccggcgtgagccgcagcagctggtggaaccagcagctcagcgacatgagctaccgccgcatgcggtgggtgcagaggaagttcatgatctacaactactgcactgacaggaagcggttcccgcagggcgtgcccgccgagtgcaagctgcgttga

SEQ ID NO: 3 - DNA, 20 bp, synthetic construct
  misc_feature 1..20: note = forward primer PheXTH2-F
  source 1..20: mol_type = other DNA
atgaggacgattgcggttgg

SEQ ID NO: 4 - DNA, 19 bp, synthetic construct
  misc_feature 1..19: note = reverse primer PheXTH2-R
  source 1..19: mol_type = other DNA
tcaacgcagcttgcactcg